(19) United States
(12) Patent Application Publication
(10) Pub. No.: US 2011/0184336 A1
(43) Pub. Date: Jul. 28, 2011

(54) DEVICE FOR PHOTODYNAMICAL THERAPY OF CANCER

(76) Inventor: Tom Kerber, Stoney Creek (CA)

(21) Appl. No.: 13/121,552

(22) PCT Filed: Sep. 29, 2009
(86) PCT No.: PCT/IL09/00929

§ 371 Date: Mar. 29, 2011

Related U.S. Application Data
(60) Provisional application No. 61/100,767, filed on Sep. 29, 2008.

(51) Int. Cl. A61M 37/00; A61N 5/06

(52) U.S. Cl. 604/20; 607/91

(57) ABSTRACT

A photodynamic therapeutic device for treating cancer is disclosed. The therapeutic device comprises at least one light emitting diode attachable to a patient's body adapted to provide an effective light fluence to a lesion area. The therapeutic device further comprises an annular structure embracing the patient's body and a passage connectable to a cooling loop providing a coolant circulative into the passage. The light emitting diode is secured to an inner surface of the annular structure. The coolant accommodated in the passage is adapted for removing heat generated by the light emitting diode.

### FIELD OF THE INVENTION

The present invention relates to a device of photodynamic therapy, and, more specifically, to a device adapted for photodynamic treating the embraceable body regions.

### BACKGROUND OF THE INVENTION

Photodynamic therapy (PDT) is also called photoradiation therapy, phototherapy, or photochemotherapy. It was first used to treat cancer over 100 years ago. It is treatment that uses drugs, called photosensitizing agents, along with light to kill cancer cells. The drugs only work after they have been activated or “turned on” by certain kinds of light.

Depending on the part of the body being treated, the photosensitizing agent is either injected into the bloodstream or put on the skin. After the drug is absorbed by the cancer cells, light is applied only to the area to be treated. The light causes the drug to react with oxygen, which forms a chemical that kills the cancer cells. PDT may also work by destroying the blood vessels that feed the cancer cells and by alerting the immune system to attack the cancer.

The period of time between when the drug is given and when the light is applied is called the drug-to-light interval. It can be anywhere from a couple of hours to a couple of days and depends on the drug used.

PDT can be used to treat some cancers, or conditions that may develop into a cancer if not treated (precancerous). It is used when the affected area or the cancer is on or near the lining of internal organs. This is usually with cancers or conditions that affect: the skin (but not melanoma), the breast, the head, the neck, the mouth, the lung, the gullet (oesophagus), the stomach, and the bile ducts.

Breast cancer affects one in eight women during their lives. Breast cancer kills more women in the United States than any cancer except lung cancer. No one knows why some women get breast cancer, but there are a number of risk factors. Risks that you cannot change include (a) age; the chance of getting breast cancer rises as a woman gets older; (b) genes; there are two genes, BRCA1 and BRCA2 that greatly increase the risk; women who have family members with breast or ovarian cancer may wish to be tested; (c) personal factors; beginning periods before age 12 or going through menopause after age 55.

Other risks include being overweight, using hormone replacement therapy, taking birth control pills, drinking alcohol, not having children or having a first child after age 35 or having dense breasts.

Symptoms of breast cancer may include a lump in the breast, a change in size or shape of the breast or discharge from a nipple. Breast self-exam and mammography can help find breast cancer early when it is most treatable. Treatment may consist of radiation, lumpectomy, mastectomy, chemotherapy, and hormone therapy.

Breast cancer recurrences after mastectomy pose a therapeutic challenge with few surgical options. If disease is localized, surgical excision can be attempted. However, these lesions often are widespread throughout the chest wall or involve heavily irradiated tissue. Many patients have received aggressive chemotherapy with little to no local response and have exhausted most avenues for local control. Multiple studies show that photodynamic therapy (PDT) provides good tumor kill for primary cutaneous malignancies and suggest its effectiveness in ablating dermal lymphatic recurrences of breast cancer. Food and Drug Administration (FDA) approved uses for PDT include lung and esophageal lesions, but treatment of bladder, head and neck, and other tumor sites with novel approaches has been reported. PDT exploits the accumulation of photosensitizers into the tumor, which then is locally excited with visible light. Selectivity of treatment comes from the excretion of drug from normal tissue over time, promoting a concentration gradient within the tumor plus the location of the activating light. Treatment depth varies with the wavelength of light that activates the sensitizer used. The singlet oxygen that is produced during the transfer of energy from light source to drug disrupts plasma, nuclear, and mitochondrial cell membranes, resulting in apoptosis. Local edema and perivascular stasis occur rapidly, within hours of treatment. Tumor necrosis can be evident within 2 to 24 hours. Photofrin (dihematoporphyrin ether; Axcan Scandipharm, Birmingham, Ala.) is the only FDA-approved photosensitizer available for the treatment of cancer. The light source used to activate Photofrin (630 nm) is topically delivered via lasers by using diffusing catheters and is focused on skin surfaces by using a microlens. This modality has been previously reported in a small number of breast cancer patients with chest wall recurrence, with good responses.

U.S. Pat. No. 6,899,723 ('723) discloses methods and compounds for PDT of a patient's target tissue, using a light source that preferably transmits light to a treatment site transcutaneously. The method provides for administering to the subject a therapeutically effective amount of a targeted substance, which is either a targeted photosensitizing agent, or a photosensitizing agent delivery system, or a targeted prodrug. This targeted substance preferably selectively binds to the target tissue. Light at a wavelength or waveband corresponding to that which is absorbed by the targeted substance is then administered. The light intensity is relatively low, but a high total fluence is employed to ensure the activation of the targeted photosensitizing agent or targeted prodrug product. Transcutaneous PDT is useful in the treatment of specifically selected target tissues, such as vascular endothelial tissue, the abnormal vascular walls of tumors, solid tumors of the head and neck, tumors of the gastrointestinal tract, tumors of the liver, tumors of the breast, tumors of the prostate, tumors of the lung, nonsolid tumors, malignant cells of the hematopoietic and lymphoid tissue and other lesions in the vascular system or bone marrow, and tissue or cells related to autoimmune and inflammatory disease.

In accordance with '723, method of therapeutically treating a target tissue provides destroying or impairing target cell by the specific and selective binding of a photosensitizer agent to the target tissue, cell, or biological component. At least a portion of the target tissue is irradiated with light at a wavelength or waveband within a characteristic absorption waveband of the photosensitizing agent. It is contemplated that an optimal total fluence for the light administered to a patient is determined clinically, using a light dose escalation trial. The total fluence administered during a treatment preferably is in the range from 500 Joules to 10,000 Joules.

It should be emphasized that the aforesaid light exposure requires light sources providing high intensity of radiation. Converting electrical energy into light in the existing light sources is characterized by intensive heat generation. Moreover, the minimal light losses are achieved under condition of attaching the light sources to the tissue that is treated. Thus, the light sources apparently have to be cooled during performing photodynamic therapy to a patient. Cooling the light sources is all the more relevant in the case of photodynamic therapy of breast cancer because of high sensitivity of the targeted tissues. Providing a cooled device of photodynamic therapy generating high-energy light fluence without heating the treated tissue is unmet and long-felt need.

### SUMMARY OF THE INVENTION

It is hence one object of the invention to disclose a photodynamic therapeutic device for treating cancer. The aforesaid device comprises at least one light emitting diode attachable to a patient's body adapted to provide an effective fluence to a lesion area.

It is a core purpose of the invention to provide the device further comprising an annular structure embracing the patient's body and a passage connectable to a cooling loop providing a circulating coolant into the passage. The light emitting diode is secured to an inner surface of the annular structure. The coolant accommodated in the passage is adapted for removing heat generated by the light emitting diode. The fluence is in the range between about 200 mw/cm² to about 3500 mw/cm².

Another object of the invention is to disclose the device adapted to be attached to a location selected from the group consisting of a breast, an arm, a leg, a neck, and any combination thereof.

A further object of the invention is to disclose embodiments of the invention which includes a device deployable on any area of the body such as the groin or abdominal wall. Furthermore, LEDs, LED clusters or segments comprising specific LED patterns of the invention could be placed in any x-y grid combination to form a flexible mat of liquid cooed segments to cover a large area.

A further object of the invention is to disclose a mode of device operation selected from the group consisting of a continuous mode, a pulse mode, an intermittent mode and any combination thereof.

A further object of the invention is to disclose the device further comprising a silicon spacer disposed between the light emitting diode and the patient's body.

A further object of the invention is to disclose the light emitting diode adapted for radiating light into a lesion area sufficient for activating administered photosensitive drugs.

A further object of the invention is to disclose the light emitting diode adapted for radiating light at a wavelength of 630 nm into a lesion area sufficient for activating administered photosensitizer 5-aminolaevulinic acid (5-ALA).

A further object of the invention is to disclose the light emitting diode adapted for radiating light at a wavelength of 585-740 nm into a lesion area sufficient for activating administered photosensitizer 5,10,15,20-tetrakis(m-hydroxyphenyl) chlorin (Foscan).

A further object of the invention is to disclose the light emitting diode adapted for radiating light at a wavelength of 570-670 nm into a lesion area sufficient for activating administered photosensitizer methyl aminolevulinate (Metvix).

A further object of the invention is to disclose the light emitting diode adapted for radiating light at a wavelength of 615-800 nm into a lesion area sufficient for activating administered photosensitizer Pd-bacteriopheophorbide (Tookad). A further object of the invention is to disclose the light emitting diode adapted for radiating light into a lesion area sufficient for activating administered Tookad (WST09) and Tookad WST11.

A further object of the invention is to disclose the light emitting diode adapted for radiating light at a wavelength of 600-750 nm into a lesion area sufficient for activating administered photosensitizers such as concentrated distillate of hematoporphyrins (Photofrin).

A further object of the invention is to disclose the light emitting diode adapted for radiating light at a wavelength of 450-600 nm into a lesion area sufficient for activating administered photosensitizer verteporfin (Visudyne).

A further object of the invention is to disclose the light emitting diode that is a plurality of emitting elements.

A further object of the invention is to disclose the emitting element that is a light emitting diode (LED).

A further object of the invention is to disclose the plurality of emitting elements distributed along the inner surface of the annular structure.

A further object of the invention is to disclose the LEDs grouped in a plurality of cooled units comprising at least two LEDs. The cooled units are distributed along the inner surface of the annular structure.

A further object of the invention is to disclose the annular structure provided adjustable according to the patient's body size.

A further object of the invention is to disclose the device further comprising a bearing surface adapted to bear a patient's body in a prone position so that the patient's breast is put in annular structure embracing thereof.

A further object of the invention is to disclose the light units are configured to be frontally attached to the patient's breast.

A further object of the invention is to disclose a method of photodynamic therapy for treating cancer. The method comprises the steps of (a) providing a device photodynamic therapeutic device for treating body cancer; the device comprises a at least one light emitting diode attachable to a patient's body adapted to provide an effective fluence to a lesion area; (b) administering an effective dose of a photosensitizer in a lesion area of the patient's body; (c) attaching the device to the patient's body; (d) beaming the patient's body with effective light fluence of a predetermined energy distribution.

It is a core purpose of the invention to provide the step of beaming performed concurrently with cooling the light emitting diode.

A further object of the invention is to disclose the device is attached to the patient's body in a location selected from the group consisting of a breast, an arm, a leg, a neck and any combination thereof.

A further object of the invention is to disclose the step of attaching the device to the patient's body further comprising a step of preliminary attaching a silicon spacer.

A further object of the invention is to disclose the step of beaming providing a substantial light intensity at a wavelength of 630 nm performed in coordination with the preceding step of administering an effective dose of a photosensitizer 5-aminolaevulinic acid (5-ALA).

A further object of the invention is to disclose the step of beaming providing a substantial light intensity at a wavelength of 585-740 nm performed in coordination with the preceding step of administering an effective dose of a photosensitizer 5,10,15,20-tetrakis(m-hydroxyphenyl) chlorin (Foscan).

A further object of the invention is to disclose the step of beaming providing a substantial light intensity at a wavelength of 570-670 nm performed in coordination with the preceding step of administering an effective dose of a photosensitizer methyl aminolevulinate (Metvix).

A further object of the invention is to disclose the step of beaming providing a substantial light intensity at a wavelength of 615-800 nm performed in coordination with the preceding step of administering an effective dose of a photosensitizer Pd-bacteriopheophorbide (Tookad).

A further object of the invention is to disclose the step of beaming providing a substantial light intensity at a wavelength of 600-750 nm performed in coordination with the preceding step of administering an effective dose of a photosensitizer concentrated distillate of hematoporphyrins (Photofrin).

A further object of the invention is to disclose the step of beaming providing a substantial light intensity at a wavelength of 450-600 nm performed in coordination with the preceding step of administering an effective dose of a photosensitizer verteporfin (Visudyne).

A further object of the invention is to disclose the step of beaming performed by a plurality of emitting elements.

A further object of the invention is to disclose the step of beaming performed by light emitting diodes (LED).

A further object of the invention is to disclose the step of beaming performed by the plurality of emitting elements distributed along an inner surface of a annular structure.

A further object of the invention is to disclose the step of beaming performed by LEDs grouped in a plurality of cooled units comprising at least two LEDs. The cooled units are distributed along the inner surface of the annular structure.

A further object of the invention is to disclose the step of attaching the device to the patient's body further comprising a step of adjusting a length of the annular structure according to a patient's body size.

A further object of the invention is to disclose the step of attaching the device to the patient's breast further comprising steps of disposing the patient on a bearing surface in a prone position and putting in the patient's breast in the annular structure so that the annular structure embraces the breast.

A further object of the invention is to disclose the step of attaching the step of attaching the device to the patient's breast performed frontally.

### BRIEF DESCRIPTION OF THE DRAWINGS

In order to understand the invention and to see how it may be implemented in practice, a plurality of embodiments is adapted to now be described, by way of non-limiting example only, with reference to the accompanying drawings, in which

FIG. 1 is an isometric view of the LED unit;

FIG. 2 is an isometric view of the photodynamic therapeutic device;

FIG. 3 is an isometric view of the photodynamic therapeutic device attached to the patient's breast;

FIG. 4 is a side view of the alternative embodiment of the photodynamic therapeutic device attached to the patient's breast;

FIG. 5 is a front view of the alternative embodiment of the photodynamic therapeutic device attached to the patient's breast;

FIG. 6 is a schematic diagram of the photodynamic therapeutic device locations on the patient's body.

FIG. 7 is a photo of the Breast liquid Phantom modelling a female breast.

FIGS. 8 and 9 are graphs of axial distribution of power density introduced into a Breast liquid Phantom.

### DETAILED DESCRIPTION OF THE INVENTION

The following description is provided, alongside all chapters of the present invention, so as to enable any person skilled in the art to make use of said invention and sets forth the best modes contemplated by the inventor of carrying out this invention. Various modifications, however, are adapted to remain apparent to those skilled in the art, since the generic principles of the present invention have been defined specifically to provide a photodynamic therapeutic device and a method of using thereof.

The term ‘photodynamic therapy (PDT)’ hereinafter refers to therapy that uses laser, or other light emitting diodes, combined with a light-sensitive drug (sometimes called a photosensitizing agent) to destroy cancer cells.

A photosensitizing agent is a drug that makes cells more sensitive to light. Once in the body, the drug is attracted to cancer cells. It does not do anything until it is exposed to a particular type of light. When the light is directed at the area of the cancer, the drug is activated and the cancer cells are destroyed. Some healthy, normal cells in the body will also be affected by PDT, although these cells will usually heal after the treatment.

About 5% to 19% of breast cancer patients suffer from chest wall recurrences after mastectomy, and these breast cancer recurrences have a high impact on physical and psychological well-being. Although surgery and radiation therapy are standard treatments for chest wall recurrences after mastectomy, PDT shows promise in treating these patients, according to the researchers.

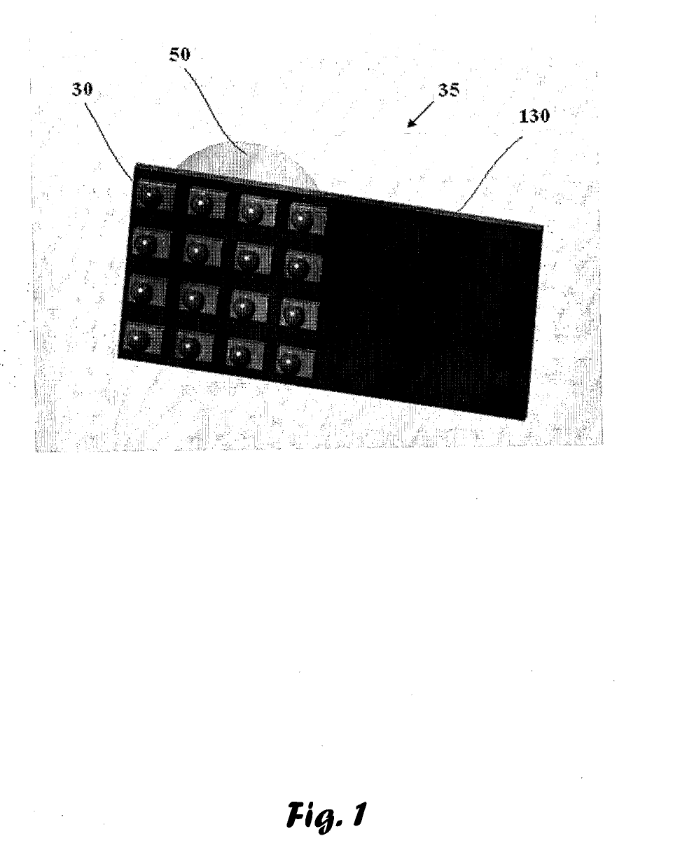

Reference is now made to FIG. 1, presenting a LED unit 35 comprising a matrix of LEDs 30 disposed on a base plate 130. A passage 50 accommodating a coolant is attached to the back of the plate 130. Thus, the heat generated by the LEDs 30 is extracted by means of the coolant circulating in the cooling loop.

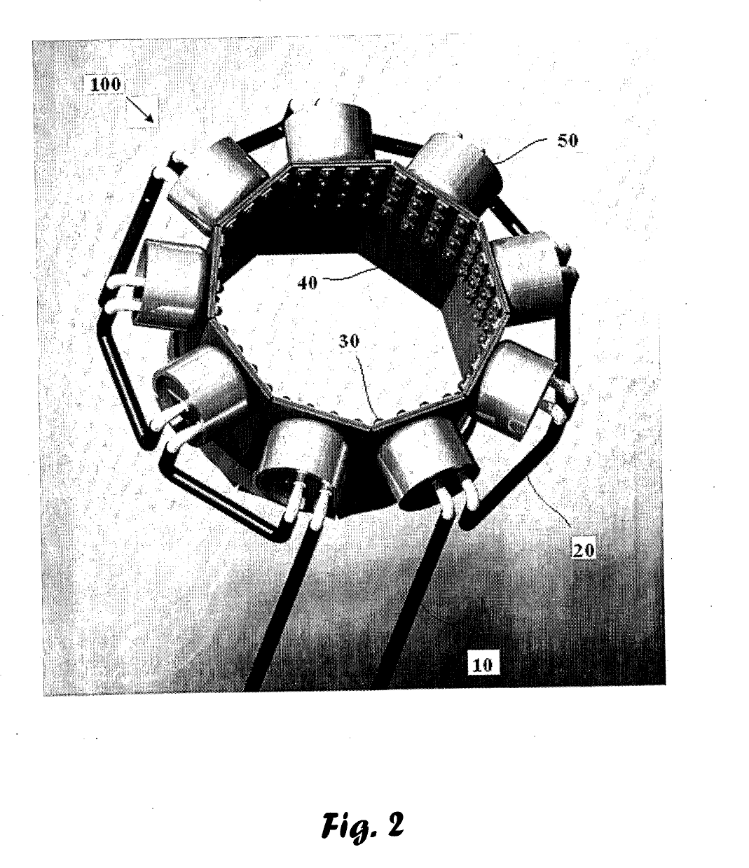

Reference is now made to FIG. 2, showing a PDT device 100 comprising an annular structure 40, LEDs 30 disposed at an inner surface of the aforesaid annual structure 40, the passages 50 accommodating the coolant circulating through feeding pipes 10 and 20. A strap 80 fixates the structure 40 on the patient's breast.

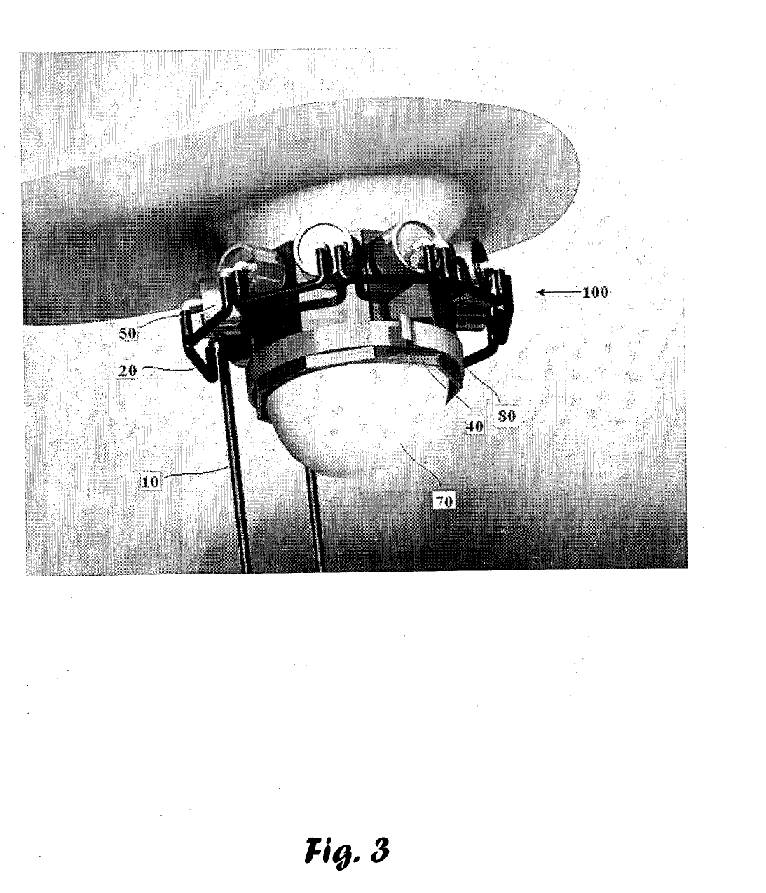

Reference is now made to FIG. 3, presenting the device 100 attached to a patient's breast 70. The light emitted by the LEDs 30 (not shown) propagates into the patient's breast 70 and affects a sensitizer concentrated in the lesion area. PDT exploits the accumulation of photosensitizers into the tumor, which then is locally excited with visible light. Selectivity of treatment comes from the excretion of drug from normal tissue over time, promoting a concentration gradient within the tumor plus the location of the activating light. Treatment depth varies with the wavelength of light that activates the sensitizer used. The singlet oxygen that is produced during the transfer of energy from light emitting diode to drug disrupts plasma, nuclear, and mitochondrial cell membranes, resulting in apoptosis. Local edema and perivascular stasis occur rapidly, within hours of treatment.

The proposed device creates high light intensity in the lesion area to provide required light fluence in shorter period of time. The heat generated by the LED is extracted by the coolant circulating in the passages 50. The proposed arrangement allows safely attaching high intensity light emitting diode to the patient's body.

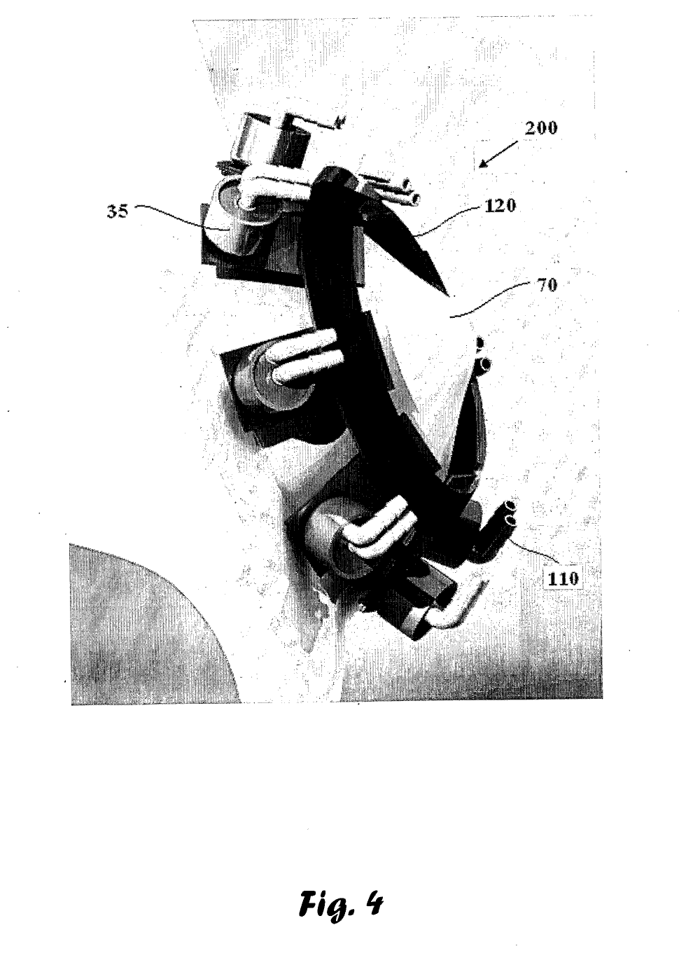

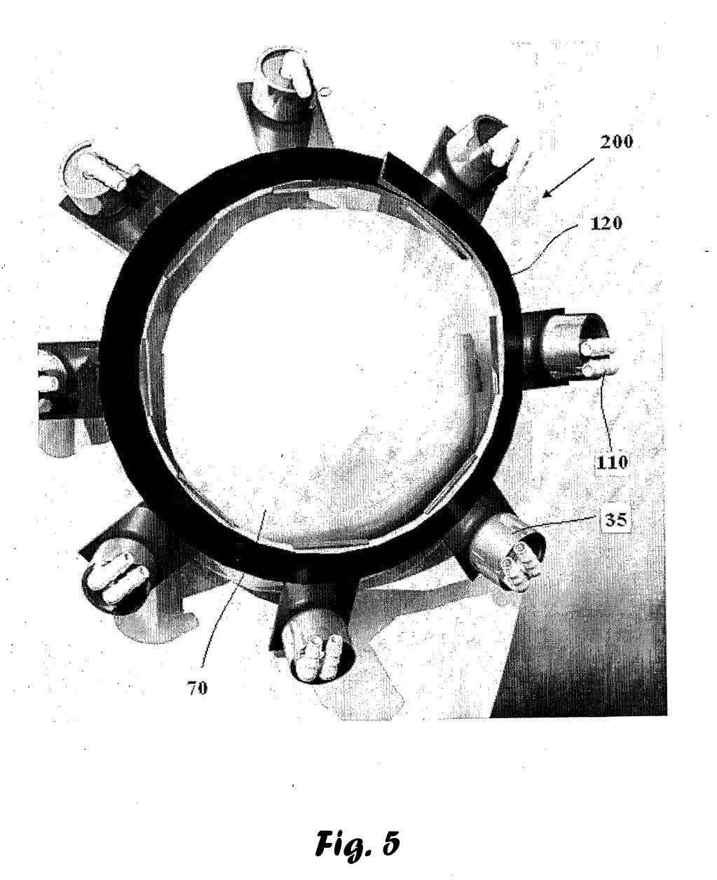

Reference is now made to FIGS. 4 and 5, shown side and front views of an alternative embodiment 200 of the PDT device, respectively. The configuration of the device 200 is conformed to a form of the patient's breast 70. The light units 35 are tilted relative to an annular structure 120. Adjustment of a length of the annular structure 120 according to a size of the patient's breast 70 is in a scope of the current invention.

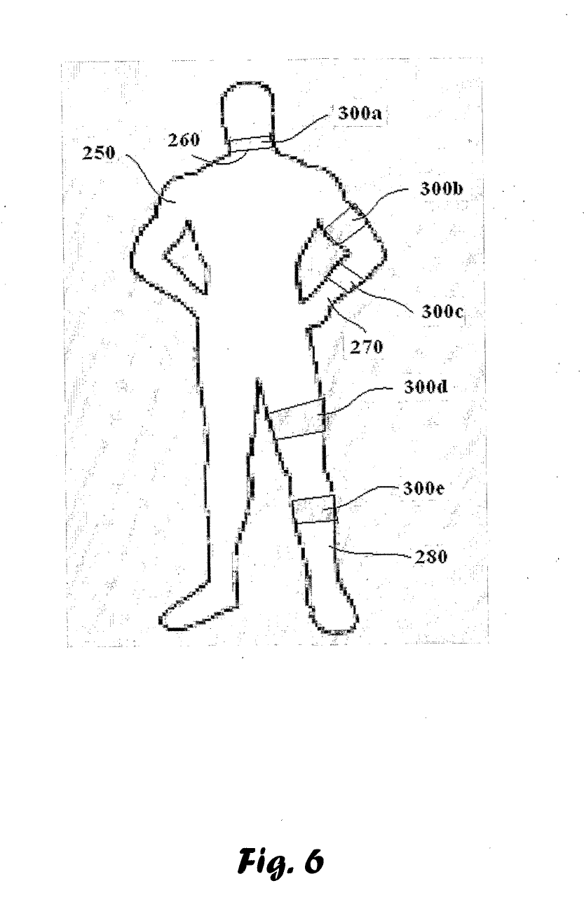

Reference is now made to FIG. 6, presenting optional body locations where the proposed therapeutic device can be attached. The therapeutic devices are attached to the patient's body 250 at a neck 260 (300a), an arm 270 (300b and 300c), and a leg 2780 (300d and 300e).

In accordance with one embodiment of the current invention, a photodynamic therapeutic device for treating cancer comprises at least one light emitting diode attachable to a patient's body adapted to provide an effective fluence to a lesion area. The aforesaid device further comprises an annular structure embracing the patient's body and a passage connectable to a cooling loop providing a circulative coolant into the passage. The light emitting diode is secured to an inner surface of the structure. The coolant accommodated in the passage is adapted for removing heat generated by the light emitting diodes. The annular embracing structure has 5 Rebel LEDs per cm. Each rebel yields 700 mw of light power. The aforesaid structure provides total of 3500 mw per cm². The LEDs have the means for internal cooling of the LEDs

The used LED (Rebel by Phillips) has a thermal heat pad area common in the prior art more powerful 100 times while keeping the operating temperature under 50 degrees C.

Our research has shown that when a light emitting diode is wrap around the breast that the light that is available at the center of the breast is a summation of the light that comes from each of the segments surrounding the breast.

90 degrees of light around a breast would yield ¼ of the fluence at the center of a breast to that which is 360 degrees (this results in 4× the light fluence) and probably adds an additional 1-2 cm of additional breast penetration depth. This is only important effect when the light fluence is high enough at the surface.

An example: 15 segments would be required to surround the breast due to the circumference of the breast (15″) and the size of each segment (1″).

The flexible “annular structure” comprises 5 to 15 small platforms or segments (hereafter referred to as segments) that are coupled together by a mechanical linkage of some type (fabric, Velcro, flexible material (silicone, etc. . . . )). Each one of the segments is small enough to cover some area say 2.5 cm×2.5 cm to say 4 cm×4 cm.

Each one of the segments is not flexible, because the very high heat removal that is required to keep the LEDs cool.

Each segment would contain 2 to 40 high power LEDs and would require up to 100 watts of heat removal for each segment.

Each segment would contain a 0.005″ thick circuit board with direct LED soldered contact copper to a liquid heat removal chamber with turbulent liquid flow to remove the maximum heat from the segment.

Each one of the segments would have a clear silicone spacer mat 0.3-0.5 cm thick directly in front of the LEDs to protect and to remove the possibility of the lens of the LED directly coming into contact with the skin and causing direct heat transfer.

A tumor requires an extremely large fluence (1000 mw/cm squared) at the surface of a breast to have enough light “effective fluence” (50 mw/cm squared) to activate the drug to treat the tumor at a distance 4 cm into the breast tissue. I think that until our patent these type of light levels described in various patents by many inventors have never been thought of for PDT.

Other patents would definitely not be able to achieve the light required to treat 3 to 6 cm deep tumors or the cooling required accomplishing the task.

An intermittent operation mode of the device is in the scope of the current invention. Intermitting active and inactive phases of illumination increases the performance of the drug because allows for cooling the skin during inactive phases to reduce heating effect of continuous light.

5ala is generally found to be effective with a dosage of 40 joules of light for surface cancer less then ½ cm depth.

40 Joules/cm2 is 40 watt seconds of light . . . 1 watt for 40 seconds over an area of 1 cm2

100 mw for 400 seconds

40 mw for 1000 seconds

Also can be seen is that a doubling of time halves the power level (light fluence) required for a successful treatment

Tripling the time requires only ⅓ of the power level

The treatment time can be increased up to 1 hour with drug still effective (beginning wait time for drug after ingestion is 4 hours).

Table 1 presents simulation results for 100, 1000 and 2500 mW/cm² on a patient's skin surface.

The first three columns show the relationship of depth to numerical constants that reflect the drop as the light penetrates the tissue.

For example, at 3 cm the light level is approximately. 33 times less then that at the surface, or 3% of the light (column 3) is present at 3 cm depth then what was at the surface.

Column 4 shows the relationship that exists when one uses a light from other patents with say 100 mw/cm².

This column shows that you would have just enough fluence with 100 mw/cm2 at the surface to treat 1 cm deep into the breast with one segment. Obviously you would not be able to treat a deep tumor with this light level.

Column 5—wrap around light (four segments) yields four times the light level.

Column 5=Column 4×four

You could only treat deeper if you wrapped the entire circumference with four segments each producing 100 mw/cm2 on a 4 cm in diameter arm (baby or small child). Each segment adds to the light at the centre core. In this example, Column 5 shows that if you use a wrap around four segment light you would be able to successfully treat to just beyond 2 cm in depth with 1000 seconds of light with 69 mw/cm2 of light fluence at the 2 cm depth.

Column 7—Represents a single segment light device yielding 1000 mw/cm2 at the surface

One can see that a single segment treatment beyond 3 cm with 4 cm depth partial light treatment. Double the time and 4 cm depth adequately treated.

Column 8—Represents a four segment light device around a circumference (yields 4× light fluence) at up to 5 cm depth treatment. 5 cm depth has 50 mw/cm2 available

Column 9—Represents a single segment 2500 mw/cm2 at the surface with a +4 cm depth of treatment 75.3 mw/cm2 available

Column 10—Represents a four segment light device (2500 mw/cm2) around a circumference at up to 6 cm depth treatment. 6 cm depth has 52 mw/cm2 available

Column 11—Represents doubling the treatment time for a four segment light device (2500 mw/cm2) around a circumference with almost a 7 cm depth treatment. 7 cm depth has 43 mw/cm2 available

Column 12—Represents tripling of the treatment time for a four segment light device (2500 mw/cm2) around a circumference with more than a 7 cm depth treatment. 7 cm depth has 65 mw/cm2 available.

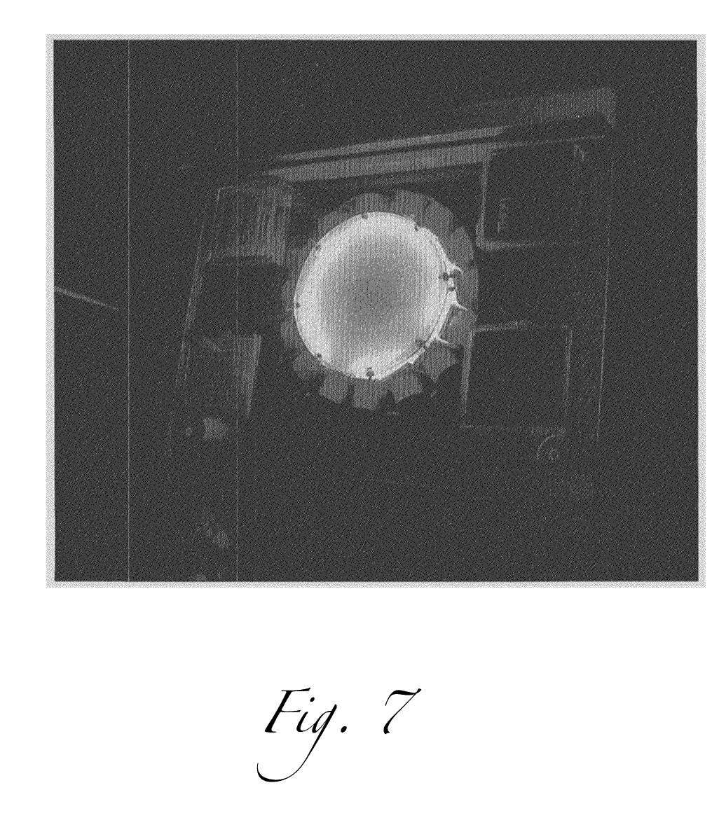

Reference is now made FIG. 7 presenting the Breast liquid Phantom modelling a female breast.

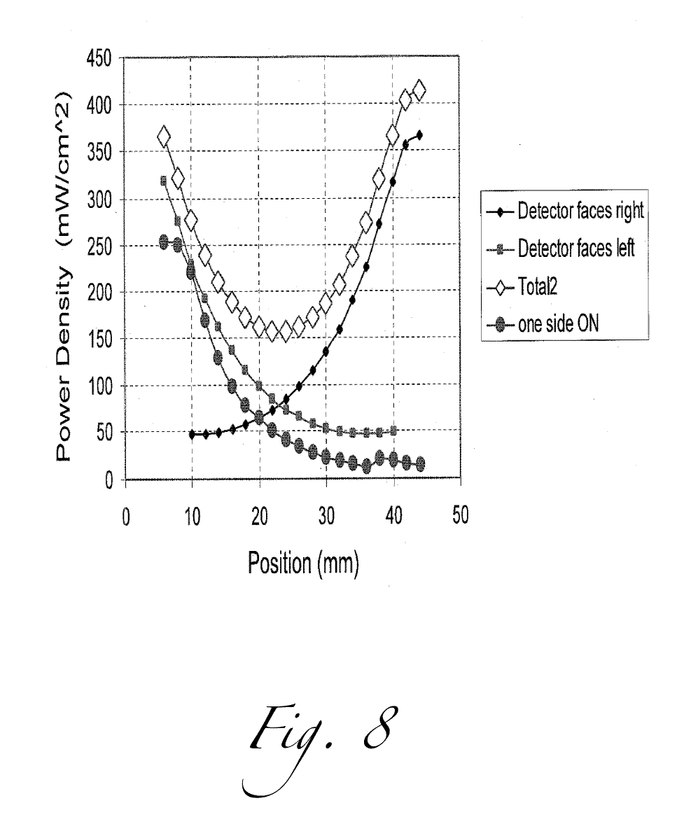

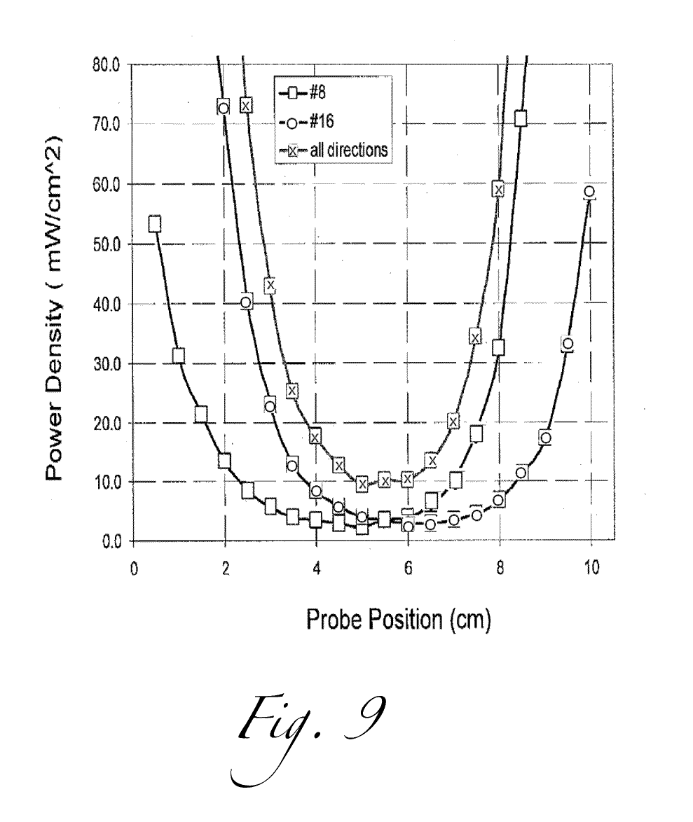

Reference is now to FIGS. 8 and 9 presenting axial distribution of power density introduced into a Breast liquid Phantom. Thermal diffusion flattens out temperature distribution.

The modelling liquid consists of a scattering agent intralipid (0.5%) and absorptive agent (black ink) characterized by absorption of 0.0006 mm⁻¹ at 630 nm.

Some embodiments of the invention utilise a coolant loop that is in series with each segment of LEDs. The series configuration reduces water flow with increased resistance and the last segments will be the hottest depending on flow rate. The aforementioned is taken into consideration during the planning of the treatment schedule.

A parallel coolant loop is also contemplated in some embodiments of the invention where greater flow rates and possibly more consistently lower temperatures are required. The parallel configuration is defined by an arrangement of the invention whereby fluid enters all segments at the same time and leaves from all segments into a larger return tube.

In some embodiments of the invention ultimate control on the light output of the LEDs on each segment is provided: the output power to the unit may be altered in 0.1% steps from 0-100%

It is another objective of the invention to disclose treatment protocols for slowly raising the power level over the treatment area.

This might be important since as one penetrates a deep area, the closest flesh to the LED segment might receive a too powerful dosage and reduce drug effectiveness. On the other hand, a continuous low output may not achieve the depth of treatment. An optimal treatment protocol may be to gradually increase the light output over treatment time, allowing each measure of depth to receive the right amount of light until that depth is treated. Light is increased for deeper penetration in staged light increases.

In accordance with another embodiment of the current invention, the device is adapted to be attached to a location selected from the group consisting of a breast, an arm, a leg, a neck, and any combination thereof.

In accordance with another embodiment of the current invention, the photodynamic therapeutic device further comprises a silicon spacer disposed between the light emitting diode and the patient's body.

In accordance with a further embodiment of the current invention, the light emitting diode is adapted for radiating light into a lesion area sufficient for activating administered photosensitive drugs.

In accordance with a further embodiment of the current invention, the light emitting diode is adapted for radiating light at a wavelength of 630 nm into a lesion area sufficient for activating administered photosensitizer 5-aminolaevulinic acid (5-ALA).

In accordance with a further embodiment of the current invention, the light emitting diode is adapted for radiating light at a wavelength of 585-740 nm into a lesion area sufficient for activating administered photosensitizer 5,10,15,20-tetrakis(m-hydroxyphenyl) chlorin (Foscan).

In accordance with a further embodiment of the current invention, the light emitting diode is adapted for radiating light at a wavelength of 570-670 nm into a lesion area sufficient for activating administered photosensitizer methyl aminolevulinate (Metvix).

In accordance with a further embodiment of the current invention, the light emitting diode is adapted for radiating light at a wavelength of 615-800 nm into a lesion area sufficient for activating administered photosensitizer Pd-bacteriopheophorbide (Tookad).

In accordance with a further embodiment of the current invention, the light emitting diode is adapted for radiating light at a wavelength of 600-750 nm into a lesion area sufficient for activating administered photosensitizer concentrated distillate of hematoporphyrins (Photofrin).

In accordance with a further embodiment of the current invention, the light emitting diode is adapted for radiating light at a wavelength of 450-600 nm into a lesion area sufficient for activating administered photosensitizer verteporfin (Visudyne).

It is acknowledged that any other combination of activating wavelengths other than those specifically mentioned herein may be provided by light emitting diodes or LEDs of the present invention, and that the means and methods of activating them are herein sufficiently disclosed, and are well within the scope of the present invention.

It is herein acknowledged that different photosensitive drugs or combinations of them may be used with some embodiments of the present invention and that the means and methods of activating them are herein sufficiently disclosed, and are well within the scope of the present invention.

In accordance with a further embodiment of the current invention, the light emitting diode is a plurality of emitting elements.

In accordance with a further embodiment of the current invention, the emitting element is a light emitting diode (LED).

In accordance with a further embodiment of the current invention, the plurality of emitting elements is distributed along the inner surface of the annular structure.

In accordance with a further embodiment of the current invention, the LEDs are grouped in a plurality of cooled units comprising at least two LEDs. The cooled units are distributed along the inner surface of the annular structure.

In accordance with a further embodiment of the current invention, a length of the annular structure is adjustable according to a patient's body size.

In accordance with a further embodiment of the current invention, the device further comprises a bearing surface adapted to bear a patient's body in a prone position so that the patient's breast is put in annular structure embracing thereof.

In accordance with a further embodiment of the current invention, the light units are configured to be frontally attached to a patient's breast.

In accordance with a further embodiment of the current invention, a method of photodynamic therapy for treating cancer is disclosed. The method comprises the steps of (a) providing a device photodynamic therapeutic device for treating body cancer; the photodynamic therapeutic device comprises an at least one light emitting diode attachable to a patient's body adapted to provide an effective fluence to a lesion area; (b) administering an effective dose of a photosensitizer in a lesion area of the patient's body; (c) attaching the aforesaid device to the patient's body; (d) beaming the patient's body with effective light fluence of a predetermined energy distribution. The step of beaming is performed concurrently with cooling the light emitting diode.

In accordance with a further embodiment of the current invention, the device is attached to the patient's body in a location selected from the group consisting of a breast, an arm, a leg, a neck and any combination thereof. It is herein acknowledged that certain embodiments of the invention may include a deployment of the device as LED clusters or segments on any area of the body such as the groin or abdominal wall. Furthermore, LEDs of the invention could be placed in any x-y grid combination to form a flexible mat of liquid cooled segments to cover a large area.

In accordance with a further embodiment of the current invention, the step of attaching the device to the patient's body further comprises a step of preliminary attaching a silicon spacer.

In accordance with a further embodiment of the current invention, the step of beaming a substantial light intensity at a wavelength of 630 nm is performed in coordination with the preceding step of administering an effective dose of a photosensitizer 5-aminolaevulinic acid (5-ALA).

In accordance with a further embodiment of the current invention, the step of beaming a substantial light intensity at a wavelength of 585-740 nm is performed in coordination with the preceding step of administering an effective dose of a photosensitizer 5,10,15,20-tetrakis(m-hydroxyphenyl) chlorin (Foscan).

In accordance with a further embodiment of the current invention, the step of beaming a substantial light intensity at a wavelength of 570-670 nm is performed in coordination with the preceding step of administering an effective dose of a photosensitizer methyl aminolevulinate (Metvix).

In accordance with a further embodiment of the current invention, the step of beaming providing a substantial light intensity at a wavelength of 615-800 nm is performed in coordination with the preceding step of administering an effective dose of a photosensitizer Pd-bacteriopheophorbide (Tookad).

In accordance with a further embodiment of the current invention, the step of beaming a substantial light intensity at a wavelength of about 615 nm to about 900 nm to radiate light into a lesion area sufficient for activating administered Tookad (WST09) and Tookad WST11.

In accordance with a further embodiment of the current invention, the step of beaming a substantial light intensity at a wavelength of 600-750 nm is performed in coordination with the preceding step of administering an effective dose of a photosensitizer concentrated distillate of hematoporphyrins (Photofrin).

In accordance with a further embodiment of the current invention, the step of beaming a substantial light intensity at a wavelength of 450-600 nm is performed in coordination with the preceding step of administering an effective dose of a photosensitizer verteporfin (Visudyne).

In accordance with a further embodiment of the current invention, the step of beaming is performed by a plurality of emitting elements.

In accordance with a further embodiment of the current invention, the step of beaming is performed by light emitting diodes (LED).

In accordance with a further embodiment of the current invention, the step of beaming is performed by the plurality of emitting elements is distributed along an inner surface of a annular structure.

In accordance with a further embodiment of the current invention, the step of beaming is performed by LEDs grouped in a plurality of cooled units comprising at least two LEDs; the cooled units are distributed along the inner surface of the annular structure.

In accordance with a further embodiment of the current invention, the step of attaching the device to the patient's body further comprises steps of adjusting a length of the annular structure according to a patient's body size.

In accordance with a further embodiment of the current invention, the step of attaching the device to the patient's breast further comprises steps of disposing the patient on a bearing surface in a prone position and putting in the patient's breast in the annular structure so that the annular structure embraces thereof.

In accordance with a further embodiment of the current invention, the step of attaching the device to the patient's breast is performed frontally.

TABLE 1
At least 40 Joules are required for activating 5ALA
(50 mW/cm2 for t = 16.7 minutes (1000 seconds))

| 1 | | | | 5 | 6 | | | | | | |
|---|---|---|---|---|---|---|---|---|---|---|---|
| d | | | 4 | x4 | d | | 8 | 9 | | 11 | 12 |
| Distance | | 3 | Most devices | peripheral | Distance | | ring form | 5 x6 LED array | 10 | double t | triple t |
| cm from | 2 | 1/2.4 exp | output | overlap | cm from | 7 | overlap | output | overlap | 33.4 | 50.1 |
| skin surface | 2.4 exp d | d | mW/cm2 | 4 | skin surface | mW/cm2 | 4 | mW/cm2 | 4 | 2 | 3 |
| 0 | 1 | 1 | 100 | | 0 | 1000 | | 2500 | | | |
| 1 | 2.400 | 0.417 | 41.667 | 166.667 | 1 | 416.667 | 1666.667 | 1041.667 | 4166.67 | 8333.33 | 12500.00 |
| 2 | 5.760 | 0.174 | 17.361 | 69.444 | 2 | 173.611 | 694.444 | 434.028 | 1736.11 | 3472.22 | 5208.33 |
| 3 | 13.824 | 0.072 | 7.234 | 28.935 | 3 | 72.338 | 289.352 | 180.845 | 723.38 | 1446.76 | 2170.14 |
| 4 | 33.178 | 0.030 | 3.014 | 12.056 | 4 | 30.141 | 120.563 | 75.352 | 301.41 | 602.82 | 904.22 |
| 5 | 79.626 | 0.013 | 1.256 | 5.023 | 5 | 12.559 | 50.235 | 31.397 | 125.59 | 251.17 | 376.76 |
| 6 | 191.103 | 0.005 | 0.523 | 2.093 | 6 | 5.233 | 20.931 | 13.082 | 52.33 | 104.66 | 156.98 |
| 7 | 458.647 | 0.002 | 0.218 | 0.872 | 7 | 2.180 | 8.721 | 5.451 | 21.80 | 43.61 | 65.41 |
| 8 | 1100.753 | 0.001 | 0.091 | 0.363 | 8 | 0.908 | 3.634 | 2.271 | 9.08 | 18.17 | 27.25 |
| 9 | 2641.808 | 0.000 | 0.038 | 0.151 | 9 | 0.379 | 1.514 | 0.946 | 3.79 | 7.57 | 11.36 |
| 1 | | | | 5 | 6 | | | | | | |
| d | | | 4 | x4 | d | | 8 | 9 | | 11 | 12 |
| Distance | | 3 | Most devices | peripheral | Distance | | ring form | 5 x6 LED array | 10 | double t | triple t |
| cm from | 2 | 1/2.6 exp | output | overlap | cm from | 7 | overlap | output | overlap | 33.4 | 50.1 |
| skin surface | 2.6 exp d | d | mW/cm2 | 4 | skin surface | mW/cm2 | 4 | mW/cm2 | 4 | 2 | 3 |
| 0 | 1 | 1 | 100 | | 0 | 1000 | | 2500 | | | |
| 1 | 2.600 | 0.385 | 38.462 | 153.846 | 1 | 384.615 | 1538.462 | 961.538 | 3846.15 | 7692.31 | 11538.46 |
| 2 | 6.760 | 0.148 | 14.793 | 59.172 | 2 | 147.929 | 591.716 | 369.822 | 1479.29 | 2958.58 | 4437.87 |
| 3 | 17.576 | 0.057 | 5.690 | 22.758 | 3 | 56.896 | 227.583 | 142.239 | 568.96 | 1137.92 | 1706.87 |
| 4 | 42.182 | 0.024 | 2.371 | 9.483 | 4 | 23.707 | 94.826 | 59.266 | 237.07 | 474.13 | 711.20 |
| 5 | 101.238 | 0.010 | 0.988 | 3.951 | 5 | 9.878 | 39.511 | 24.694 | 98.78 | 197.55 | 296.33 |
| 6 | 242.971 | 0.004 | 0.412 | 1.646 | 6 | 4.116 | 16.463 | 10.289 | 41.16 | 82.31 | 123.47 |
| 7 | 583.129 | 0.002 | 0.171 | 0.686 | 7 | 1.715 | 6.860 | 4.287 | 17.15 | 34.30 | 51.45 |
| 8 | 1399.511 | 0.001 | 0.071 | 0.286 | 8 | 0.715 | 2.858 | 1.786 | 7.15 | 14.29 | 21.44 |
| 9 | 3358.826 | 0.000 | 0.030 | 0.119 | 9 | 0.298 | 1.191 | 0.744 | 2.98 | 5.95 | 8.93 |

1-33. (canceled)

34. A photodynamic therapeutic device for treating cancer; said device comprising at least one light emitting diode attachable to a patient's body adapted to provide an effective fluence to a lesion area ranging between about 200 mw/cm² to about 3500 mw/cm²; wherein said device further comprises an annular structure embracing said patient's body and a passage connectable to a cooling loop providing a circulating coolant into said passage; said diode is secured to an inner surface of said structure; said coolant accommodated in said passage is adapted for removing heat generated by said diode; said fluence is in the range between about 200 mw/cm² to about 3500 mw/cm²

35. The photodynamic therapeutic device according to claim 34, wherein a mode of device operation is selected from the group consisting of a continuous mode, a pulse mode, an intermittent mode and any combination thereof.

36. The photodynamic therapeutic device according to claim 34, wherein said device is adapted to be attached to a location selected from the group consisting of a breast, an arm, a leg, a neck, and any combination thereof.

37. The photodynamic therapeutic device according to claim 34, wherein said device further comprises a transparent silicon spacer disposed between said diode and said patient's body.

38. The photodynamic therapeutic device according to claim 34, wherein said diode is adapted for radiating light into a lesion area sufficient for activating administered photosensitive drugs; said light emitting diode is adapted for radiating light at least one wavelength selected from the group consisting of: about 630 nm into a lesion area sufficient for activating administered photosensitizer 5-aminolaevulinic acid (5-ALA); about 585 to about 740 nm into a lesion area sufficient for activating administered photosensitizer 5,10,15,20-tetrakis(m-hydroxyphenyl) chlorin (Foscan); about 570 to about 670 nm into a lesion area sufficient for activating administered photosensitizer methyl aminolevulinate (Metvix); about 615 to about 800 nm into a lesion area sufficient for activating administered photosensitizer Pd-bacteriopheophorbide (Tookad). about 600 to about 750 nm into a lesion area sufficient for activating administered photosensitizer concentrated distillate of hematoporphyrins (Photofrin); about 450 to about 600 nm into a lesion area sufficient for activating administered photosensitizer verteporfin (Visudyne) and any combination thereof.

39. The photodynamic therapeutic device according to claim 34, wherein said light emitting diode is a plurality of emitting elements.

40. The photodynamic therapeutic device according to claim 34, wherein said plurality of emitting elements is distributed along said inner surface of said annular structure.

41. The photodynamic therapeutic device according to claim 34, wherein said LEDs are grouped in a plurality of cooled light units comprising at least two LEDs; said cooled units are distributed along said inner surface of said annular structure; a length of said annular structure is adjustable according to a patient's body size.

42. The photodynamic therapeutic device according to claim 34, wherein said device further comprises at least one element selected from the group consisting of a bearing surface adapted to bear a patient's body in a prone position so that said patient's breast is put in annular structure embracing thereof and said light unit configured to be frontally attached to a patient's breast.

43. A method of photodynamic therapy for treating cancer; said method comprises the steps of
(a) providing a device photodynamic therapeutic device for treating cancer; said device comprises a at least one light emitting diode attachable to a patient's body adapted to provide an effective fluence to a lesion area;
(b) administering an effective dose of a photosensitizer in a lesion area of said patient's body;
(c) attaching said device to said patient's body;
(d) beaming said patient's body with effective light fluence of a predetermined energy distribution;
wherein said step of beaming is performed concurrently with cooling said light emitting diode.

44. The method according to claim 43, wherein said device is attached to said patient's body in a location selected from the group consisting of a breast, an arm, a leg, a neck and any combination thereof.

45. The method according to claim 43, wherein a mode of device operation is selected from the group consisting of a continuous mode, a pulse mode, an intermittent mode and any combination thereof.

46. The method according to claim 43, wherein said step of attaching said device to said patient's body further comprises a step of preliminary attaching a silicon spacer.

47. The method according to claim 43, wherein said step of beaming at maximum light intensity at least one wavelength selected from the group consisting of: a wavelength of about 630 nm performed in coordination with said preceding step of administering an effective dose of a photosensitizer 5-aminolaevulinic acid (5-ALA); a wavelength of about 585 to about 740 nm performed in coordination with said preceding step of administering an effective dose of a photosensitizer 5,10,15,20-tetrakis(m-hydroxyphenyl) chlorin (Foscan); a wavelength of about 570 to about 670 nm is performed in coordination with said preceding step of administering an effective dose of a photosensitizer methyl aminolevulinate (Metvix); a wavelength of about 615 to about 800 nm performed in coordination with said preceding step of administering an effective dose of a photosensitizer Pd-bacteriopheophorbide (Tookad); a wavelength of about 600 to about 750 nm performed in coordination with said preceding step of administering an effective dose of a photosensitizer concentrated distillate of hematoporphyrins (Photofrin); a wavelength of about 450 to about 600 nm performed in coordination with said preceding step of administering an effective dose of a photosensitizer verteporfin (Visudyne) and any combination thereof.

48. The method according to claim 43, wherein said step of beaming is performed by said plurality of emitting elements is distributed along an inner surface of an annular structure.

49. The method according to claim 48, wherein said step of beaming is performed by LEDs grouped in a plurality of cooled units comprising at least two LEDs; said cooled units are distributed along said inner surface of said annular structure.

50. The method according to claim 43, wherein said step of attaching said device to said patient's breast further comprises a step of adjusting a length of said annular structure according to a patient's breast size.

51. The method according to claim 43, wherein said step of attaching said device to said patient's breast further comprises steps of disposing said patient on a bearing surface in a prone position and putting in said patient's breast in said annular structure so that said annular structure embraces thereof and/or attaching said device to said patient's breast is performed frontally
